(19) United States
(12) Patent Application Publication
(10) Pub. No.: US 2007/0161054 A1
(43) Pub. Date: Jul. 12, 2007

(54) Methods and compositions for targeting secretory lysosomes

(76) Inventors: Daniel Rajotte, Ridgefield, CT (US); Alisa Kabcenell, Ridgefield, CT (US)

Correspondence Address: MICHAEL P. MORRIS;BOEHRINGER INGELHEIM CORPORATION, 900 RIDGEBURY ROAD, P O BOX 368, RIDGEFIELD, CT, 06877-0368, US

(21) Appl. No.: 11/610,235

(22) Filed: Dec. 13, 2006

Related U.S. Application Data
(63) Continuation of application No. 10/637,887, filed on Aug. 8, 2003, now abandoned.
(60) Provisional application No. 60/403,464, filed on Aug. 14, 2002.

(51) Int. Cl. G01N 33/567; C12N 9/26

(52) U.S. Cl. 435/7.2; 435/325; 435/320.1; 435/201

(57) ABSTRACT

This invention relates to methods and compositions for targeting proteins to secretory lysosomes. The invention further provides methods of use in drug screening assays, and methods of purifying secretory lysosomes.

### RELATED APPLICATION

This application is a continuation of U.S. application Ser. No. 10/637,887, filed Aug. 8, 2003, which claims, as does the present application, priority to U.S. Provisional Application Ser. No. 60/403,464, filed Aug. 14, 2002.

### BACKGROUND

This invention relates to methods and compositions for targeting proteins to secretory lysosomes. The invention further provides methods of use in drug screening assays, and methods of purifying secretory lysosomes.

Mast cells are specialized secretory cells that release a variety of biologically active substances. Mast cells are found resident in tissues throughout the body, particularly in association with structures such as blood vessels, nerves, and in proximity to surfaces in contact with the external environment (see Metcalfe et al. Physiol Rev. 77:1033-1079, 1997). Mast cell activation may be initiated upon interaction of a multivalent antigen with its specific IgE antibody attached to the cell membrane via its high affinity receptor, FcεRI. Mast cells and basophils play a central role in inflammatory and allergic reactions (see Williams, et al. J. Allergy Clin. Immunol. 105:847-859, 2000). They are known to release potent inflammatory mediators such as histamine, proteases, chemotactic factors and metabolites of arachidonic acid that act on the vasculature, smooth muscle, connective tissue, mucous glands and inflammatory cells.

The pathways for granule biogenesis and exocytosis in mast cells are still largely obscure (Griffiths, G. M. et al., Biochem Biopys Res Commun. 222(3):802-808, 1996; Masuda et al., FEBS Lett. 470:61-64, 2000; Baram, D. et al., J Immunol. 167(7):4008-4016, 2001). Mast cells contain structures known as secretory lysosomes which are a mixture of lysosomes and secretory granules (Stinchcombe and Griffiths, J Cell Biol. 146(1):1-6, 1999). The mast cell granule can be described as a modified lysosome, specialized for fusion with the plasma membrane, and with other lysosomal granules, after receptor activation. Although similar secretory lysosomes are found in hematopoietic cells, little is known about the mechanisms by which these organelles receive and deliver their cargo. For example, Riesbeck et al. (WO 98/42850) disclose protein targeting to endothelial cell Weibel-Palade bodies. Weibel-Palade bodies contain the adhesion molecule P-selectin.

There are two categories of inflammatory mediators in mast cells and basophils, preformed mediators and newly formed mediators. Preformed mediators, stored in the cytoplasmic granules of rodent or human mast cells, include histamine, proteoglycans, cytokines, serine proteases, carboxypeptidase A and small amounts of sulfatases and exoglycosidases (Metcalfe et al., Physiol Rev. 77(4):1033-1079, 1997). Histamine acts on a set of receptors (H1, H2, H3, H4) on cells and tissues and is rapidly metabolized extracellularly. Proteoglycans may function to package histamine and basic proteins into secretory granules, and in human mast cells may stabilize the protease tryptase. Neutral proteases, which account for the vast majority of the granule protein, serve as markers of mast cells. Newly generated mediators, often absent in resting mast cells, consist of arachidonic acid metabolites, principally leukotriene C and prostaglandin D. These mediators are typically produced during IgE receptor activation. Of particular interest in humans is the production of tumor necrosis factor (TNF), Interleukin (IL)-4, IL-5 and IL-6.

Proteases are the major protein constituent exocytosed from activated mast cells (Huang et al., J Clin Immunol. 18:169-183, 1998). Tryptases, chymases, and carboxypeptidases are the three major families of proteases stored in the secretory granules of mast cells. Chymases are part of the serine protease family. Immunohistochemical localization indicates that they are only synthesized in mast cells (Beil et al., Histol Histopathol. 15(3):937-946, 2000). Human, primate, and dog chymase generate angiotensin II (Ang II) from Ang I, while mouse and rat chymases degrade Ang II (Fukami et al., Curr Pharm Des. 4(6):439-453, 1998). Chymase also degrades extracellular matrix, and processes procollagenase, inflammatory cytokines and other bioactive peptides. As a result, chymase plays important roles in inflamed tissues through its proteolytic activities.

In human cells, genes encoding two chymotryptic enzymes (chymase and Cathepsin G-like protease) and one mast cell carboxypeptidase enzyme and at least two genes encoding tryptase peptides have been detected. The gene encoding chymase is closely linked to the gene encoding cathepsin G, an enzyme apparently expressed in mast cells, and to the genes encoding granzymes. Mucosal (MC)-type mast cells contain tryptase, chymase, cathepsin G-like protease and mast-cell carboxypeptidase. The biological function of mast cell neutral proteases, like mast cells themselves, remains to be fully clarified. For example, on-going mast cell activation in asthma appears to be a characteristic of this chronic inflammatory disease.

In murine mast cells, five chymases (Mouse Mast Cell Protease (MMCP)-1, -2, -3, -4, and -5), one mast cell carboxypeptidase and two tryptases (MMCP-6 and -7) have been reported. In rodents, the protease composition of mast cell subsets differs. In rats two isoforms of chymase, Rat Mast Cell Protease (RMCP) I (Lagunoff and Pritzl Arch Biochem Biophys. 173(2):554-563, 1976) and RMCP II (Kido et al, Arch Biochem Biophys. 239(2):436-443, 1985) were found to distinguish the mast cells in mucosal surfaces (RMCP-II positive), from other mast cells (RMCP-I positive) (Gibson and Miller Immunology 58(1):101-104, 1986). More recently, two additional serine proteases were isolated by PCR amplification from rat serosal MC: the rat tryptase (the counterpart of MMCP-6) and an additional chymase named RMCP III (Lutzelschwab et al., J Exp Med. 185(1):13-29, 1997). The latter protease is the rat counterpart of mouse MMCP-5.

Scientists have reported on a protein, Rab37, that can localize to the surface of mast cell granules when fused to green fluorescent protein (GFP) and is over-expressed in bone-marrow derived mast cells (see Masuda et al., FEBS Lett. 470:61-64, 2000). Rab37 appears to localize to the cytoplasmic surface of granules. However, Masuda does not teach the use of Rab37 to target granules.

Until the present invention, there have been no reports on the use of a targeting moiety to localize proteins to secretory lysosomes. Current state of the art employs indirect methods for the detection of granule content or exocytotic activity. For example, mast cell granules have been studied by monitoring their content with antibodies, or measuring the activity of enzymes such as hexosaminidase (see Schwartz et al. J. Immunol. 123:1445-1450, 1979; and Dragonetti et al. J. Cell Sci. 113:3289-3298, 2000). Other indirect methods relate proteins and their functions to other secretory compartments such as the endoplasmic reticulum, Golgi and trans Golgi network (see Donaldson and Lippincott-Schwartz, Cell 101:693-696, 2000). Therefore, a targeting moiety localizing proteins to the inner core of mast cell secretory lysosomes is a significant advancement in mast cell research and drug discovery.

Current methods that attempt to quantify the release of mediators upon degranulation of cells containing secretory lysosomes are lengthy and costly (see, for example, Schwartz et al., J Immunol. 123:1445-1450, 1979 and Schulman et al., J Immunol. 131:2936-1941, 1983). The present invention overcomes these obstacles such that not only is the content of secretory lysosomes quantified, but also the movement of secretory lysosomes is monitored in real time.

Until the present invention, no methods were amenable for High Throughput Screening (HTS) to screen for modulators of secretory lysosomes. Current state of the art does not allow direct monitoring of cell degranulation in an HTS setting. For example Demo et al. (Cytometry, 36(4):340-8, 1999) disclose assays that require several biochemical measurements and many steps that consume time, energy and likely have low reproducibility due to the reagents used (histamine, tryptase, hexosaminidase). For example, histamine is known to have a poor dynamic range for quantification in HTS setting. Other methods have been used to monitor cell degranulation using fluorescent probes such as acridine orange (see Love Histochemistry 62:221-225, 1979) but these assays lack specificity for the exocytotic process and poor-signal to noise ratios (see Demo et al. Cytometry, 36(4):340-8, 1999). One group has disclosed a quantitative measurement of cell degranulation using a flow cytometric annexin-V binding assay (see Demo et al. Cytometry, 36(4):340-8, 1999). Flow cytometry however, is not amenable to HTS format.

Current art provides protocols to obtain subcellular fractions enriched in mast cell granules by fractionation of cell homogenates on Percoll or sucrose gradients (Lindmark et al., J Leukocyte Biol. 66:634-643, 1994). Advantageously, the present invention provides methods to purify secretory lysosomes to a higher degree than currently achieved. The increased level of purification achieved by the present invention is crucial for proteomics studies aimed at the identification of novel drug discovery targets involved in cell activation.

### BRIEF SUMMARY

The present invention relates to methods for localizing secretory lysosomes in real-time within a cell using a targeted molecule. Such methods are useful for many purposes, including but not limited to, studying secretory lysosome movement and fusion, directly monitoring exocytosis, developing cellular screens for cell activation and purification of secretory lysosomes.

Prior to the present invention, there were no existing direct methods of targeting protein to secretory lysosomes. Mast cell secretory lysosomes are specialized organelles that contain proteases, heparin, histamine and several cytokines. Previously, secretory lysosomes were studied by indirect methods such as monitoring their content with antibodies or measuring the activity of enzymes such as hexosaminidase.

The present invention relates to a secretory targeting fusion moiety comprising a polypeptide that specifically localizes to a secretory lysosome and a label polypeptide. The present invention also relates to a secretory targeting fusion moiety comprising a nucleotide sequence encoding a polypeptide that specifically localizes to a secretory lysosome and a nucleotide sequence encoding a label polypeptide.

The present invention further relates to a secretory targeting fusion moiety comprising a protease selected from the group consisting of tryptases, chymases, and carboxypeptidases, preferably Mouse Mast Cell Protease (MMCP)-1, -2, -3, -4, -5, -6, and -7; Rat Mast Cell Protease (RMCP) I and RMCP II; human chymases; human tryptases; Cathepsin G-like protease; Cathepsin G; carboxypeptidase A; and hexosaminidase; more preferably RMCP II.

The invention further related to a cell comprising a secretory targeting fusion moiety of the present invention. In an embodiment, the label polypeptide is a fluorescent molecule. In a specific embodiment, the fluorescent molecule is Discosoma sp. red fluorescent protein or green fluorescent protein. In an embodiment, the cell of the invention is selected from the group consisting of: mast cells, basophils, hemopoietic cells, melanocytes, and goblet cells. In a specific embodiment, the cell is a mast cell.

In a particular embodiment, the invention relates to a cell line expressing a secretory lysosome targeting fusion moiety as deposited with the American Type Culture Collection and assigned accession number PTA-4571.

In one embodiment of the present invention, there is disclosed a method for detecting and quantifying degranulation comprising: (a) incubating a cell expressing a secretory lysosome targeting fusion moiety comprising a label molecule in the presence of a cell activator; (b) incubating the cell expressing the secretory lysosome targeting fusion moiety comprising a label molecule in the absence of the cell activator; and (c) detecting and quantifying the release of label in the supernatant in the presence of the cell activator compared to the release of label in the supernatant in the absence of the cell activator, wherein an increase in the release of label in the supernatant in the presence of the cell activator indicates degranulation.

In another embodiment of the present invention, there is disclosed a method for detecting and quantifying inhibition of degranulation comprising: (a) incubating a cell expressing a secretory lysosome targeting fusion moiety comprising a label molecule with a cell activator in the presence of a test substance; (b) incubating the cell expressing the secretory lysosome targeting fusion moiety comprising a label molecule with the cell activator in the absence of the test substance; and (c) detecting and quantifying a change in the release of label in the supernatant in the presence of the test substance compared to the release of label in the supernatant in the absence of test substance, wherein a decrease in the release of label in the supernatant in the presence of test substance indicates inhibition of degranulation.

In another embodiment of the present invention, there is disclosed a method for detecting and quantifying degranulation at the single cell level comprising: (a) incubating a cell expressing a secretory lysosome targeting fusion moiety comprising a label molecule in the absence of a cell activator; (b) detecting and quantifying the amount of label in the absence of a cell activator, (c) incubating the cell of step (a) in the presence of a cell activator; (d) detecting and quantifying the amount of label in the presence of the cell activator; and (e) detecting a change in the amount of label in the cell in the presence of the cell activator compared to the amount of label in the cell in the absence the cell activator, wherein a decrease in the amount of label indicates degranulation.

In another embodiment of the present invention, there is disclosed a method for detecting and quantifying degranulation at the single cell level comprising: (a) incubating a cell expressing a secretory lysosome targeting fusion moiety comprising a label molecule in the presence of a cell activator; (b) detecting and quantifying the amount of label in the presence of the cell activator, (c) incubating the cell of step (a) in the presence of the cell activator and a test substance; (d) detecting and quantifying the amount of label in the presence of the cell activator and the test substance; and (e) comparing the amount of label in the cell in the presence of the test substance to the amount of label in the cell in the absence the test substance, wherein an increase in the amount of label in the presence of the test compound indicates degranulation.

### BRIEF DESCRIPTION OF THE FIGURES

FIG. 1. Stable expression of RMCP-DsREd in RBL-2H3 cells. Cells were transfected by electroporation with the RMCP-DsRED vector (“DsRED”, Discosoma sp. red fluorescent protein). Cells were then submitted to Geneticin® (G-418, GIBCO Invitrogen Corp., Carlsbad, Calif.) selection for 10 days. FIG. 1 shows FACS analysis of control RBL-2H3 population compared to the selected pool of cells expressing RMCP-DsRED.

FIG. 2. FACS analysis of the clone RBL-RMCP/2C2. Individual clones from the cellular pool expressing RMCP-DsRED were generated. This figure shows FACS analysis of the RBL-2H3 parental cells compared to the clone RBL-RMCP/2C2.

FIG. 3. Stable expression of RMCP-DsRED recombinant protein in RBL-2H3 cells. RBL-2H3 cells were stably transfected with an expression vector for the DsRED protein alone or the RMCP-DsRED fusion protein. After the selection process, individual clones were analyzed by confocal microscopy to determine subcellular distribution of the recombinant protein. A) Shows confocal image of cells expressing the DsRED protein and its cytoplasmic expression. B) Shows a confocal image of cells expressing the RMCP-DsRED. fusion protein. The punctate fluorescence correlates with the localization of granules or secretory lysosomes in mast cells or basophils.

FIG. 4. The RMCP-DsRED construct targets secretory lysosomes and granules in RBL 2H3 cells. Cells stably expressing the RMCP-DsRED fusion protein were treated with the LysoTracker® probe. Left panel shows intracellular localization of secretory lysosomes and granules using the LysoTracker® probe. Middle panel shows the distribution of the RMCP-DsRED fusion protein. The overlay of LysoTracker® and RMCP-DsRED images (right panel) shows colocalization of the RMCP-DsRED with the secretory lysosome and granule compartments.

FIG. 5. RMCP-DsRED is released upon IgE stimulation of RBL 2H3 cells. RBL 2H3 cells stably expressing the RMCP-DsRED protein (clone RBL-RMCP/2C2) were stimulated with IgE and antigen (DNP-HSA). Hexosaminidase and histamine are two granule markers that are released from the cells within minutes after stimulation. Quantification of the fluorescence released after stimulation shows a rapid release of the RMCP-DsRED fusion protein followed by a slower phase of release 90 minutes after stimulation.

FIG. 6. Flow cytometric analysis of fluorescent granules from RBL-2H3 clones stably expressing RMCP-DsRED protein. Cells expressing DsRED alone (control) or RMCP-DsRED were submitted to subcellular fractionation on a Percoll gradient. The fraction containing the secretory lysosome and granules was then analyzed by organelle flow cytometry. Cells expressing DsRED (left panel) showed no fluorescent lysosomes or granules compared to cells expressing RMCP-DsRED (right panel).

FIG. 7. Purification of fluorescent secretory lysosomes and granules using FACS sorting. Fluorescently labeled secretory lysosomes and granules gated in FIG. 6 (right panel) were sorted by flow cytometry and assayed for hexosaminidase content. Hexosaminidase specific activity is shown for the post nuclear supernatant (PNS), the lysosomal/granule fraction isolated by Percoll gradient and for the FACS sorted material.

FIG. 8. Live cell imaging of RBL-2H3 cells expressing RMCP-DsRED following IgE stimulation. Live RBL-RMCP/2C2 cells were visualized by confocal microscopy. Cells were imaged at time 0 and then stimulated with IgE and antigen (DNP-HSA). Cells were incubated for a total of 2 hours and images were taken at various time points. The 15 min., 1 h and 2 h time points are shown.

### DETAILED DESCRIPTION OF THE INVENTION

1. Definitions

The following definitions are provided to facilitate understanding of certain terms used herein:

As used herein and in the appended claims, the singular forms “a”, “an”, and “the” include plural reference unless the context clearly dictates otherwise.

By “cells” it is meant to include cells in any form, including, but not limited to, cells retained in tissue, cell clusters and individually isolated cells.

By “cell line” it is meant cells capable of stable growth in vitro for many generations.

By “clone” it is meant a population of cells derived from a single cell or common ancestor by mitosis.

By “degranulation” it is meant movement and exocytosis of secretory lysosomes. By “polypeptide” it is meant peptide or protein and variants thereof.

By “secretory lysosome” it is meant a dual-function organelle that is used as both the lysosome (for degradation) and for storage of secretory proteins of the cell and which shares many features with both conventional lysosomes and secretory granules, such as structure and content.

By “secretory lysosome targeting fusion moiety” it is meant a moiety comprising: (a) a polypeptide that specifically localizes to a secretory lysosome or a nucleotide sequence encoding such polypeptide and (b) a label polypeptide or nucleotide sequence encoding such label polypeptide.

By “secretory lysosome targeting moiety” it is meant a polypeptide that specifically localizes to a secretory lysosome or a nucleotide sequence encoding such polypeptide.

By “variant” it is meant a sequence, such as a polypeptide, that differs from another sequence, but retains essential properties thereof, that is, properties for which the sequence is utilized in its application (e.g., protease activity). For example, a variant of a polypeptide may differ in amino acid sequence by one or more substitutions, additions, and deletions from the reference polypeptide. By “variant” it is also meant to include fragments of a full length sequence that retain essential properties thereof.

2. The Methods and Constructs

The present invention overcomes many of the problems associated with prior art methods for detection and monitoring of secretory lysosome content and exocytotic activity. By transfecting cells with a moiety comprising a nucleotide sequence encoding a secretory lysosome-specific protein and a nucleotide sequence encoding a label molecule, the present invention permits the study of the movement of secretory lysosomes and the release of their contents in real time and secretory lysosome quantification in living cells.

Cells that may be used in the present invention include cell lines and primary cells that have secretory lysosomes, including but not limited to mast cells, basophils, hemopoietic cells, melanocytes, and goblet cells. In a preferred embodiment, the cells are mast cells.

In one embodiment, a cell line expressing a secretory lysosome targeting moiety is designated RBL-RMCP/2C2 (accession no. PTA-4571, deposited on Aug. 7, 2002 with the American Type Culture Collection, 10801 University Boulevard, Manassas, Va. 200110-2209 under the terms of the Budapest Treaty).

The secretory lysosome targeting moiety of the present invention localizes in secretory lysosomes. The secretory lysosome targeting moiety of the present invention may comprise constituents of secretory lysosomes such as proteases, for example, tryptases, chymases and carboxypeptidases including but not limited to: Mouse Mast Cell Protease (MMCP)-1, -2, -3, -4, -5, -6, and -7; Rat Mast Cell Protease (RMCP) I and RMCP II; human chymases; human tryptases; Cathepsin G-like protease; Cathepsin G; carboxypeptidase A; hexosaminidase; or polypeptides thereof or polypeptides encoded by related genes or orthologues. In an embodiment, the secretory lysosome targeting moiety comprises RMCP II.

The secretory lysosome targeting moiety of the present invention can further comprise any polypeptide of interest or nucleotide sequence encoding such polypeptide. In the screening and detection methods of the present invention, the polypeptide or nucleotide sequence encoding such polypeptide is a label, preferably a fluorescent molecule, e.g., Discosoma sp. red fluorescent protein (DsRED) or green fluorescent protein (GFP).

A label molecule can include, but is not limited to, a luminescent molecule (e.g. luciferase), an enzyme (e.g. horse radish peroxidase, β-galactosidase), a fluorescent molecule (e.g., Discosoma sp. red fluorescent protein (DsRED) or green fluorescent protein (GFP).

In the therapeutic methods of the invention described below, the secretory lysosome targeting moiety can further comprise any therapeutic polypeptide or nucleotide sequence encoding such polypeptide of interest, including but not limited to enzymes, cytokines, growth factors, and recombinant antibodies (single chains).

The present invention also provides methods for identifying compounds that modulate degranulation using the secretory lysosome targeting fusion moieties of the invention. For example, a cell of the invention expressing a secretory lysosome targeting fusion moiety comprising a label molecule is incubated with a cell activator in the presence and absence of a test substance. A change in the release of fluorescence in the supernatant in the presence of the test substance would indicate that the test substance modulates degranulation, for example, an increase in the release of fluorescence in the supernatant indicates degranulation. In a preferred embodiment, the secretory lysosome targeting fusion moiety comprises a fluorescent label molecule.

Cell activators include but are not limited to: IgE and a multivalent antigen, phorbol myristate acetate (PMA), ionomycin, compound 48/80, toll-like receptors, and protease receptors. In one embodiment of the invention cell activators are selected from the group consisting of: IgE and a multivalent antigen, phorbol myristate acetate (PMA), and ionomycin.

In another embodiment, the present invention provides methods for increasing the purity of secretory lysosome preparations using the secretory lysosome targeting fusion moieties.

Preferably, a cell line transfected with a secretory lysosome targeting fusion moiety comprising a fluorescent molecule is fractionated by methods known in the art (e.g., Percoll or sucrose gradient) to obtain subcellular fractions enriched in secretory lysosome. The secretory lysosome-rich fraction is then further purified using fluorescence activated cell sorting (FACS).

In another embodiment, the present invention provides methods for studying secretory lysosome maturation, biosynthesis, cell differentiation, migration and activation in vivo using a secretory lysosome targeting moiety further comprising a reporter gene.

In another embodiment, the present invention provides methods for studying and quantifying exocytosis (degranulation) in real time, at a single cell level using a secretory lysosome targeting fusion moiety, preferably comprising a fluorescent label molecule, wherein detection and quantification is performed using fluorescence or confocal microscopy, for example, using a Cellomics ArrayScan® System (Cellomics, Inc., Pittsburgh, Pa.). In these methods, for example, fluorescence would be detected before and after stimulation of a cell transfected with a fluorescent marker and a secretory lysosome targeting moiety, wherein a reduction in fluorescence indicates degranulation. These methods, therefore can be used to screen for compounds that inhibit the release of secretory lysosomes.

In another embodiment, the present invention provides methods for delivery of therapeutic polypeptides in vivo using a secretory lysosome targeting fusion moiety comprising a therapeutic polypeptide. In one embodiment, a cell line transfected with the therapeutic secretory lysosome targeting fusion moiety is encased in an immunoisolation device and implanted in a subject.

The screening methods of the present invention may be adapted to High Throughput Screening (HTS) and Ultra High Throughput Screening (UHTS). The HTS and UHTS can employ, for example, a Zymark Allegro™ modular robotic system (Zymark Corp., Hopkinton, Mass.) to dispense reagents, buffers, and test compounds into either 96-well or 384-well black microtiter plates (from Dynex (Dynex Technologies, Denkendorf, Germany) or Corning (Corning Costar, Cambridge, Mass.), respectively).

### EXAMPLES

The following examples are provided to illustrate the invention, but not to limit its scope. Other variants of the invention will be readily apparent to one of ordinary skill in the art.

### Example 1

### Cloning of RMCP II in the Expression Vector pDsRED1-N1

The sequence encoding RMCP II was retrieved from GenBank (accession no. J02712; Benfey et al. JBC 262:5377, 1987). RMCP II was cloned by polymerase chain reaction (PCR). The rat basophilic leukemia (RBL-2H3) cell line (accession no. CRL-2256, American Type Culture Collection (ATCC), Manassas, Va.) was used as a source of RNA. The reverse transcriptase reaction (first strand cDNA synthesis) was done using the Superscript™ amplification system by GIBCO BRL (catalog no. 18089-011, GIBCO BRL, Invitrogen Corp., Carlsbad, Calif.) according to the manufacturer's instructions. The PCR amplification was performed on first strand cDNA (from previous step) using the following oligos:

(SEQ ID NO:1)

5′-TCAGATCTCGAGATGCAGGCCCTACTATTCCTG-3′
and

(SEQ ID NO:2)

5′-CTGCAGAATTCGGCTACTTGTATTAATGACTGCAT-3′.

The PCR conditions were as follows: 94° C. (30 sec)-62° C. (30 sec)-72° C. (50 sec). The PCR product was purified and digested with the restriction enzymes XhoI and EcoRI. These restriction sites are provided by the oligos. The fragment was then cloned in the same sites of the pDsRED 1-N 1 vector from Clontech (catalog no. 6921-1, BD Biosciences Clontech, Palo Alto, Calif.). This cloning strategy results in the full length RMCP II cDNA in-frame with the N-terminus of the cDNA for the fluorescent protein DsRED. The sequence identity of the recombinant vector, RMCP-DsRED, was confirmed by DNA sequencing.

### Example 2

### Stable Expression of RMCP-DsRED Recombinant Protein in RBL-2H3 Cells

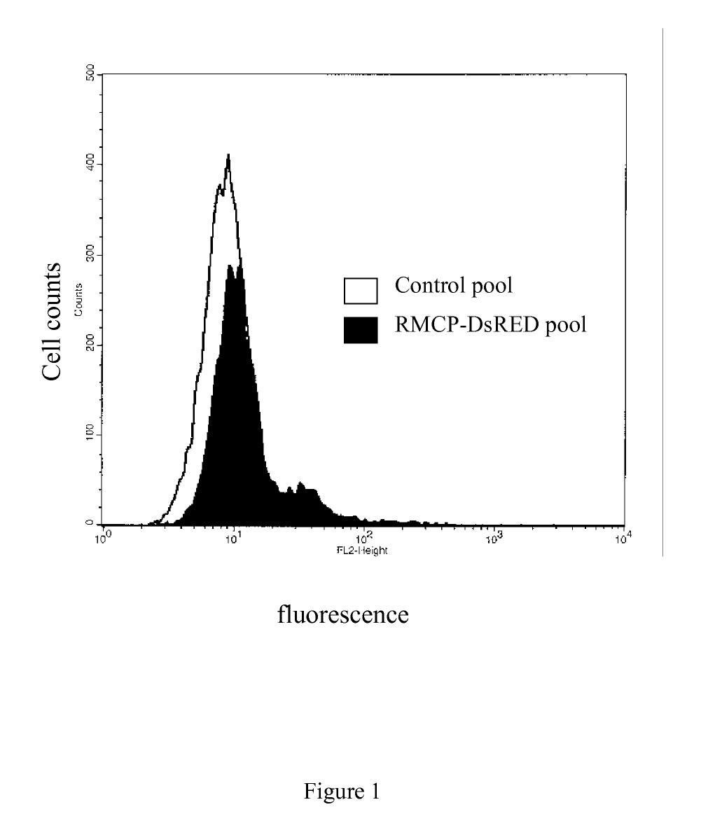

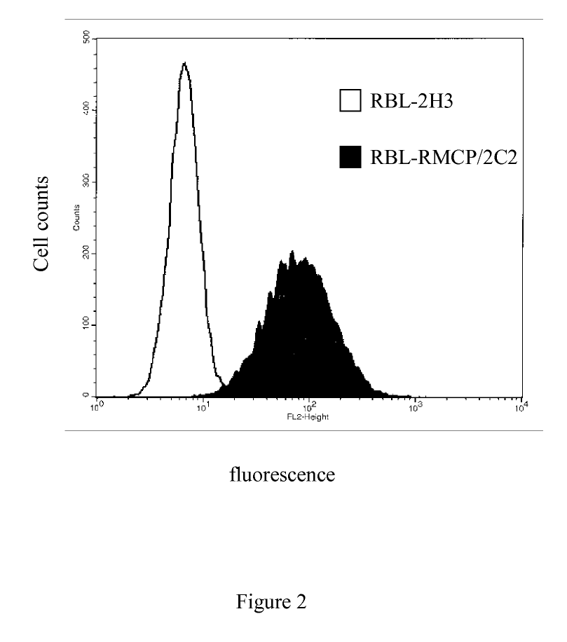

RBL-2H3 cells (8×10⁶) were transfected with the RMCP-DsRED vector (45 μg) by electroporation (Guillemot et al. JCB 110:2215-2225, 1997). The conditions for the electroporation were 300 V and 960 μF in a volume of 800 μl. The transfected cell line is designated RBL-RMCP/2C2. Transfected cells were transferred to the appropriate culture media and incubated for 48 h. Positive clones were then selected by the addition of 1 mg/ml of active Geneticin® (GIBCO BRL, Invitrogen Corp., Carlsbad, Calif.). Ten days after transfection, the cells were analyzed by fluorescence activated cell sorting (FACS). As seen in FIG. 1, a population of cells positive for red fluorescence was detected. Individual clones from the cell population were isolated by FACS and amplified. As seen in FIG. 2, RBL-RMCP/2C2 is a positive clone expressing the RMCP-DsRED fusion protein.

### Example 3

### Expression of RMCP-DsRED Fusion Protein in Granules

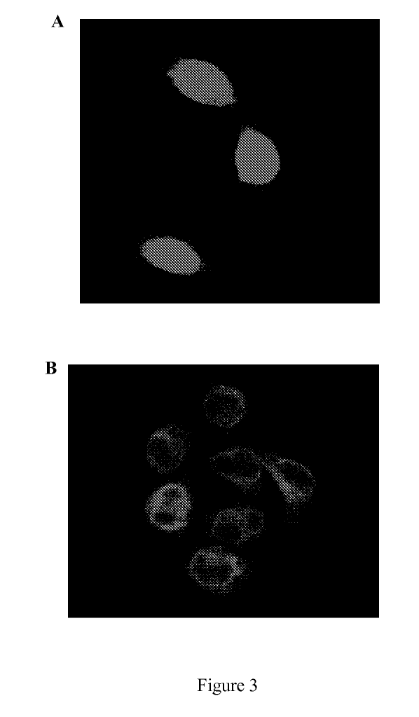

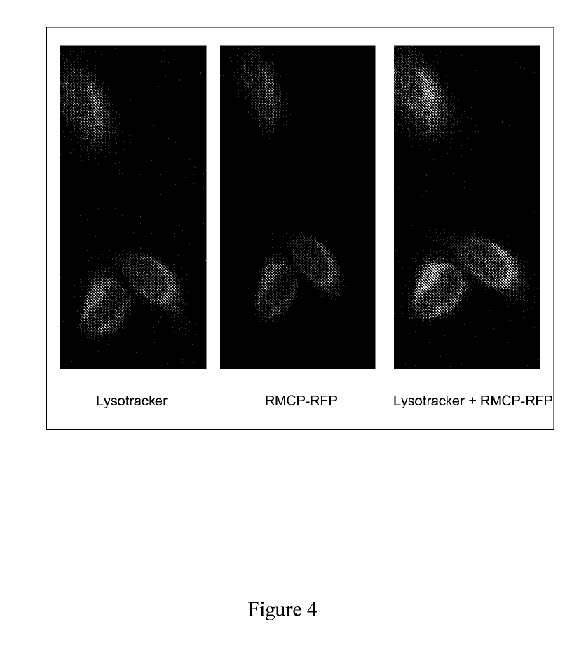

The subcellular localization of the RMCP-DsRED protein from the RBL-RMCP/2C2 clone was analyzed by confocal microscopy. A cellular clone expressing the pDsRED vector alone was used as control. As shown in FIG. 3, cells transfected with the pDsRED control vector expressed the DsRED protein in the cytoplasm (diffuse fluorescence). In contrast, RMCP-DsRED fusion shows punctuate expression in proximity to the plasma membrane. This pattern correlates with the localization of granules in mast cells or basophils. The LysoTracker® probe (Molecular Probes, catalog no. L-7526) is a weakly basic amine that selectively accumulates in cellular compartments with low internal pH which include lysosomes and dense core granules (Sreyer, J. A. et al. Nature 388:474-478, 1997). As seen in FIG. 4, in the RBL-RMCP/2C2 clone, the LysoTracker® probe co-localizes with the RMCP-DsRED fusion protein. This result confirmed that the RMCP-DsRED fusion protein is targeted to granules.

### Example 4

### Release of Red Fluorescence Upon Cell Activation

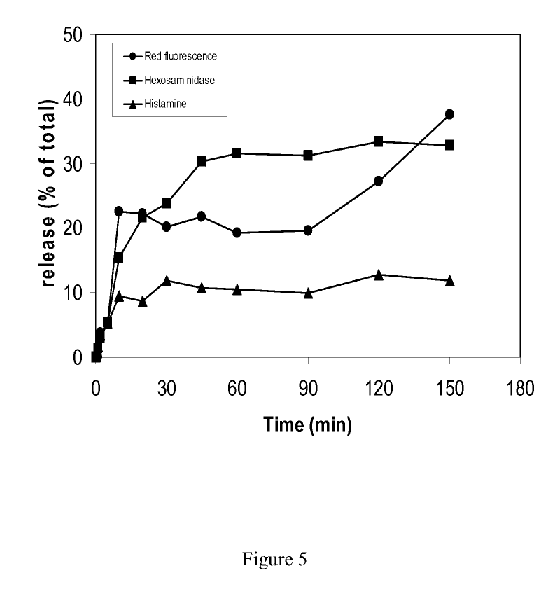

Mast cells and basophils respond to IgE and antigen stimulation by the rapid release (within minutes) of their granule content into the extracellular milieu. A standard assay in the field is to stimulate the RBL-2H3 cell line with an antigen-specific IgE molecule and then cross-link the IgE receptor by the addition of the corresponding antigen (Roa M. et al. J. Immunol. 159:2815-2823, 1997). The release of histamine and β-hexosaminidase are typically used as markers to monitor degranulation (Schwartz L. B. et al. J. Immunol. 123:1445-1450, 1979). As expected, when the RBL-RMCP/2C2 cells are stimulated with a mouse anti-DNP (dinitrophenyl) IgE followed by stimulation with DNP-HSA (DNP antigen coupled to human serum albumin) they release both histamine and β-hexosaminidase within minutes (FIG. 5). As shown in FIG. 5, the RBL-RMCP/2C2 cells also release red fluorescence upon stimulation. The fluorescence release was measured from the supernatant of the cell culture (in a 96-well plate format) at the indicated time using an LJL fluorescence plate reader. The kinetics of histamine, β-hexosaminidase and fluorescence release are the same.

### Example 5

### Purification of Granules by FACS

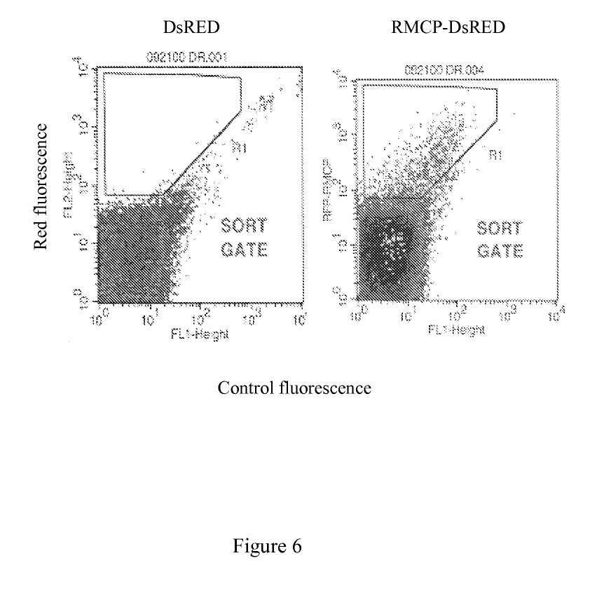

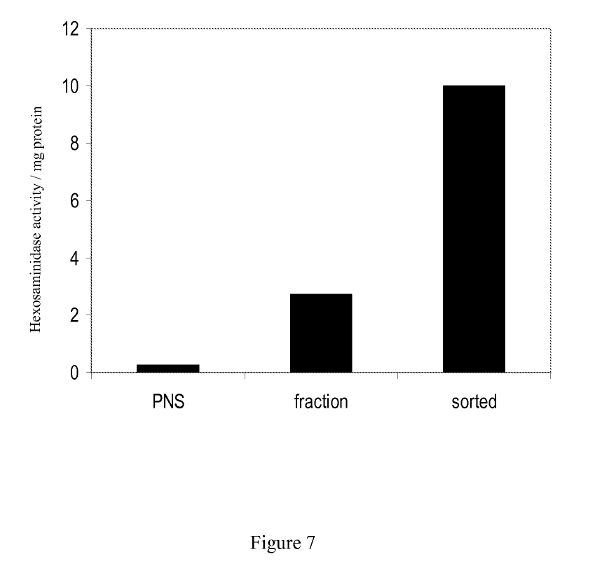

Cell homogenates were fractionated on Percoll or sucrose gradients to obtain subcellular fractions enriched in mast cell granules (Kruger P. G. et al., Exp. Cell Res. 129:83-93, 1980). Using the RBL-RMCP/2C2 cell line, the granule-rich fraction was further purified by FACS. As shown in FIG. 6, a distinct fluorescent population was detected in the granule fraction isolated from RBL-RMCP/2C2 cells as compared to a control cell line. The positive (gated) population was then separated from the total fraction by organelle sorting (Fialka I. et al. J. Biol. Chem. 274:26233-26239, 1999). As shown in FIG. 7, hexosaminidase specific activity was substantially increased after sorting. Thus, this step contributed to a substantial increase in the purity of the granule fraction.

### Example 6

### Live Imaging of Cells Expressing RMCP-DsRED Following IgE and Antigen Stimulation

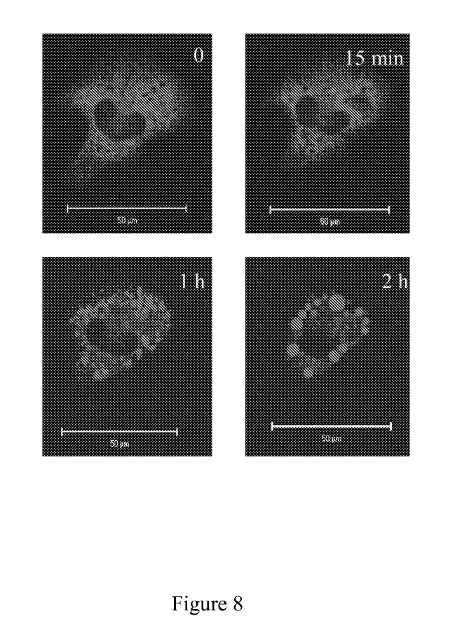

Live RBL-RMCP/2C2 cells were visualized by confocal microscopy. Cells were sensitized with anti-DNP IgE, imaged at time 0 and then stimulated with antigen (DNP-HSA). Cells were incubated for a total of 2 hours and images were taken at various time points. The 15 min, 1 h and 2 h time points are shown in FIG. 8. As shown in FIG. 8, degranulation can be observed in real time at the single cell level.

### Example 7

### Quantifying the Inhibition of Degranulation by a Test Substance in RBL-RMCP/2C2 cells

Cells are seeded in 96-well plates at a density of 2×10⁴ cells per well and incubated overnight. Cells are then washed twice in culture media and incubated for 2 hours at 37° C. in culture media containing the test substance (concentrations ranging from 1 mM to 1 pM) and 1 μg/ml of anti-DNP IgE monoclonal antibody (SPE7 clone, Sigma). The cells are then washed twice in Tyrode's buffer (10 mM Hepes, pH 7.4, 130 mM NaCl, 5 mM KCl, 1.4 mM CaCl₂, 1 mM MgCl₂, 5.6 mM glucose, and 0.1% BSA) and then stimulated with 100 ng/ml DNP-HSA (Sigma) in Tyrode's buffer for one hour. Aliquots (100 μl) from the culture supernatants are analyzed for release of red fluorescence. The red fluorescence is detected on an LJL Bioanalyst (LJL Biosystems, Sunnyvale, Calif.) set at 530 nm for excitation and 580 nm for emission.

All publications and patents cited herein are incorporated by reference in their entireties.

What is claimed is:

1. A secretory lysosome targeting fusion moiety comprising: (a) a polypeptide that specifically localizes to a secretory lysosome and (b) a label polypeptide.

2. A secretory lysosome targeting fusion moiety comprising: (a) a nucleotide sequence encoding a polypeptide that specifically localizes to a secretory lysosome and (b) a nucleotide sequence encoding a label polypeptide.

3. The secretory lysosome targeting fusion moiety according to claim 2 wherein the polypeptide that specifically localizes to a secretory lysosome comprises a protease selected from the group consisting of: tryptases, chymases and carboxypeptidsases.

4. The secretory lysosome targeting fusion moiety according to claim 3 wherein the protease is selected from the group consisting of: Mouse Mast Cell Protease (MMCP)-1, -2, -3, -4, -5, -6, and -7; Rat Mast Cell Protease (RMCP) I and RMCP II; human chymases; human tryptases; Cathepsin G-like protease; Cathepsin G; carboxypeptidase A; and hexosaminidase.

5. The secretory lysosome targeting fusion moiety according to claim 4 wherein the protease is Rat Mast Cell Protease (RMCP) II according to GenBank accession no. J02712.

6. The secretory lysosome targeting fusion moiety according to claim 2 wherein the label polypeptide is a fluorescent molecule.

7. A cell comprising a secretory lysosome targeting fusion moiety according to claim 2.

8. The cell according to claim 7 wherein the label polypeptide is a fluorescent molecule.

9. The cell according to claim 8 wherein the fluorescent molecule is Discosoma sp. red fluorescent protein or green fluorescent protein.

10. The cell according to claim 7, wherein the cell is selected from the group consisting of: mast cells, basophils, hemopoietic cells, melanocytes, and goblet cells.

11. The cell according to claim 10, wherein the cell is a mast cell.

12. A cell line expressing a secretory lysosome targeting fusion moiety as deposited with the American Type Culture Collection and assigned accession number PTA-4571.

13. A method for detecting and quantifying degranulation comprising: (a) incubating a cell expressing a secretory lysosome targeting fusion moiety comprising a label molecule in the presence of a cell activator; (b) incubating the cell expressing the secretory lysosome targeting fusion moiety comprising a label molecule in the absence of the cell activator; and (c) detecting and quantifying the release of label in the supernatant in the presence of the cell activator compared to the release of label in the supernatant in the absence of the cell activator, wherein an increase in the release of label in the supernatant in the presence of the cell activator indicates degranulation.

14. A method for detecting and quantifying inhibition of degranulation comprising: (a) incubating a cell expressing a secretory lysosome targeting fusion moiety comprising a label molecule with a cell activator in the presence of a test substance;
(b) incubating the cell expressing the secretory lysosome targeting fusion moiety comprising a label molecule with the cell activator in the absence of the test substance; and (c) detecting and quantifying a change in the release of label in the supernatant in the presence of the test substance compared to the release of label in the supernatant in the absence of test substance, wherein a decrease in the release of label in the supernatant in the presence of test substance indicates inhibition of degranulation.

15. The method according to claim 13 wherein the cell expressing a secretory lysosome targeting fusion moiety is selected from the group consisting of: mast cells, basophils, hemopoietic cells, melanocytes, and goblet cells.

16. The method according to claim 13 wherein the cell expressing a secretory lysosome targeting fusion moiety is a mast cell.

17. The method according to claim 13 wherein the label molecule is a fluorescent molecule.

18. The method according to claim 17 wherein the fluorescent molecule is Discosoma sp. red fluorescent protein or green fluorescent protein.

19. The method according to claim 13 wherein the secretory lysosome targeting fusion moiety comprises a nucleotide acid sequence encoding a protease selected from the group consisting of: tryptases, chymases and carboxypeptidases.

20. The method according to claim 13 wherein the protease is selected from the group consisting of: Mouse Mast Cell Protease (MMCP)-1, -2, -3, -4, -5, -6, and -7; Rat Mast Cell Protease (RMCP) I and RMCP II; human chymases; human tryptases; Cathepsin G-like protease; Cathepsin G; carboxypeptidase A; and hexosaminidase.

21. The method according to claim 13 wherein the protease is Rat Mast Cell Protease (RMCP) II.

22. The method according to claim 13 wherein the cell line expressing the secretory lysosome targeting fusion moiety is the cell line as deposited with the American Type Culture Collection and assigned accession number PTA-4571.

23. The method according to claim 13 wherein the cell activator is selected from the group consisting of: IgE and a multivalent antigen, phorbol myristate acetate, ionomycin, compound 48/80, toll-like receptors, and protease receptors.

24. The method according to claim 13 wherein the cell activator is selected from the group consisting of: IgE and a multivalent antigen, phorbol myristate acetate, and ionomycin.
